(19) United States
(12) Patent Application Publication
(10) Pub. No.: US 2012/0124889 A1
(43) Pub. Date: May 24, 2012

(54) FUNGI FOR ODOR CONTROL

(75) Inventor: Jarrod E. Leland, Blacksburg, VA (US)

(73) Assignee: NOVOZYMES BIOLOGICALS, INC., Salem, VA (US)

(21) Appl. No.: 13/275,476

(22) Filed: Oct. 18, 2011

Related U.S. Application Data
(60) Provisional application No. 61/408,155, filed on Oct. 29, 2010.

(51) Int. Cl. A01M 1/10; A01N 59/14; A01N 43/90; A01H 15/00; A01N 43/54; A01N 43/88; A01N 43/50; A01P 7/04; A61L 9/01; A01N 43/56

(52) U.S. Cl. 43/107; 424/76.5; 800/295

(57) ABSTRACT

The present invention is directed to a composition and method thereof for reducing or eliminating malodors produced by decaying arthropod cadavers comprising an effective amount of one or more entomopathogenic fungi. The invention provides a novel way to eliminate the malodors stemming from pest control methods as traps and chemical pesticides are frequently used to control pests and pest associated diseases in restaurants, commercial hotels, motels, and residential housing.

### CROSS-REFERENCE TO RELATED APPLICATIONS

This application claims the benefit under 35 U.S.C. 119 of U.S. provisional application No. 61/408,155 filed Oct. 29, 2010, the contents of which are fully incorporated herein by reference.

### REFERENCE TO A DEPOSIT OF BIOLOGICAL MATERIAL

This application contains a reference to a deposit of biological material, which deposit is incorporated herein by reference. For complete information see the detailed description of the invention.

### FIELD OF THE INVENTION

The present invention relates to insect killing compositions comprising entomopathogenic fungi and use of such compositions for reducing or eliminating odors emitted by dead insects.

### BACKGROUND OF THE INVENTION

Pest infestation is a common problem in households and industrial settings. Many products are available for controlling arthropod pests such as insects and for preventing new infestations. However, one common problem associated with pest control is the unpleasant odor that remains after the death and further decay of the pest bodies. These unpleasant odors can be caused by the initial release of substances referred to as “necromones” or fatty acid substances released upon the death of many pests including cockroaches and caterpillars. Other odors can be caused by the release of gasses from the natural decay of the dead insects through autolysis and putrefaction.

Solutions for controlling pest populations are well known. Common methods target pests using chemical or natural insecticides either alone or in combination with one another. For example, U.S. Pat. No. 5,888,989 discloses insecticidal and acricidal compositions of silafluofen and at least one entomopathogenic fungus for protection against pests, in particular, agricultural pests.

U.S. Pat. No. 5,057,315 discloses the use of entomopathogenic fungi as a non-toxic alternative for controlling cockroach populations.

U.S. Pat. No. 5,679,362 is directed to an insect infection chamber capable of attracting insects and infecting them with viable pathogenic Metarhizium spores.

While solutions such as traps and chemical pesticides are frequently used to control pests and pest associated diseases in restaurants, commercial hotels, motels, and residential housing, it is desirable to eliminate odors resulting from the pest control as well. In short, a need exists to rid of malodors associated with the decay of insect cadavers.

### SUMMARY OF THE INVENTION

The present invention provides a composition comprising one or more entomopathogenic fungi capable of reducing or eliminating malodor resulting from dead pests, alone or in combination with a commercially available chemical pesticide.

It is yet another object of the present invention to provide a method for horizontally transmitting one or more entomopathogenic fungi across a population of arthropods known to exhibit semi-social behaviour by coupling a chemical pesticide to the entomopathogenic fungi such that the chemical pesticide will provide a fast kill but allow the resulting spores from the cadaver to infect the surviving population of arthropods.

In one embodiment, it is an object of the present invention to provide a composition for reducing or eliminating malodors produced by decaying arthropod cadavers including an effective amount of one or more entomopathogenic fungi. The composition may include one or any of number of entomopathogenic fungi, either alone or in combination, with other fungi. The genus of entomopathogenic fungi may include, but are not limited to fungi from the genera Metarhizium spp., Beauveria spp., Paecilomyces spp, Lecanicillium spp., or Hirsutella spp. In particular, the entomopathogenic fungus of the composition is Metarhizium anisopliae. More particularly the entomopathogenic fungus is DSM 3884, DSM 3885, or a mixture thereof.

The composition for reducing or eliminating malodors produced by a dead arthropod may further include an effective amount of a chemical pesticide. The chemical pesticide may be, but is not limited to, a bait formulation, a sprayable formulation, or a dustable formulation. Further still, the active ingredient for the chemical pesticide may be, but is not limited to, boric acid, abamectin, fipronil, hydramethylnon, indoxacarb, and imidacloprid.

In another embodiment, it is an object of the present invention to provide a method for reducing or eliminating malodors produced by decaying arthropod cadavers comprising by preparing a composition having an effective amount of one or more entomopathogenic fungi and exposing that composition to a target arthropod pest. The composition may include one or any of number of entomopathogenic fungi, either alone or in combination, with other fungi. The genus of entomopathogenic fungi may include, but are not limited to fungi from the genus Metarhizium spp., Beauveria spp., Paecilomyces spp, Lecanicillium spp., or Hirsutella spp. In particular, the entomopathogenic fungus of the composition is Metarhizium anisopliae. More particularly the entomopathogenic fungus is DSM 3884, DSM 3885, or a mixture thereof.

It is envisioned that the arthropod pest will be exposed to the entomopathogenic composition through methods including, but not limited to, placing the composition in a trap, combining the composition with a food source, combining the composition with a chemical pesticide, or any feasible combination thereof. It is further envisioned that the chemical pesticide may be, but is not limited to, a bait formulation, a sprayable formulation, or a dustable formulation. It is further envisioned that the active ingredient for the chemical pesticide may be, but is not limited to, boric acid, abamectin, fipronil, hydramethylnon, indoxacarb, and imidacloprid.

While it is envisioned that the composition and method described herein is intended to target all arthropod pest, it is also envisioned that the invention will be particularly useful in combating the malodors associated with decaying cockroach cadavers. More particularly, it is envisioned that composition and method described herein will be particularly useful in combating the malodors associated with the decay of German cockroach Blatella germanica, brown banded cockroach Supella longipaloa, Oriental cockroach Blatta orientalis, smoky brown cockroach Periplaneta fuliginosa, American cockroach Periplaneta Americana, Turkenstan cockroach Blatta lateralis, and field cockroach Blatta vaga cadavers.

### BRIEF DESCRIPTION OF THE DRAWINGS

FIG. 1 is a graphical representation illustrating the percent mortality of cockroaches subjected to various treatments over time.

FIG. 2 is a Gas Chromatography-Mass Spectrometer (GC-MS) reading of a blank sample without cockroach cadavers.

FIG. 3 is a GC-MS reading of volatiles produced from cockroach cadavers killed by a commercially available chemical cockroach bait.

FIG. 4 is a GC-MS reading of volatiles produced from cockroach cadavers killed by a commercially available chemical cockroach bait and sporulating with Met52.

FIGS. 5A-5B are graphical representations illustrating the percent sporulation and percent mortality of different species of cockroaches subjected to various treatments over time.

FIGS. 6A-6B are bar graph representations illustrating the correlation between percent sporulation and percent mortality for B. germanica cockroaches in the presence and absence of food over time.

FIGS. 7A-7B are bar graph representations illustrating the correlation between percent sporulation and percent mortality for B. orientalis cockroaches in the presence and absence of food over time.

FIG. 8 is a bar graph representation illustrating the correlation between percent sporulation and percent mortality for B. germanica cockroaches subjected to a horizontal transmission assay.

### DETAILED DESCRIPTION OF THE INVENTION

The present invention relates to compositions and methods thereof for reducing and or eliminating odors associated with the chemical and natural death of insect and arthropod pests.

### Deposit of Biological Material

The following biological material has been deposited with the Deutsche Sammlung von Mikroorganismen (DSM), Grisebachstraβe, D-3400 Göttingen, Bundersrepublik Deutschlang, under the terms of the Budapest Treaty on the International Recognition of the Deposit of Microorganisms for the Purposes of Patent Procedure, and have been given the following accession numbers:

| | Accession | |
|---|---|---|
| Deposit | Number | Date of Deposit |
| Metarhizium anisopliae | DSM 3884 | Oct. 24, 1986 |
| Metarhizium anisopliae | DSM 3885 | Oct. 24, 1986 |

The strains have been deposited under conditions that assure that access to the culture will be available during the pendency of this patent application to one determined by foreign patent laws to be entitled thereto. The deposits represent a substantially pure culture of the deposited strain. The deposits are available as required by foreign patent laws in countries wherein counterparts of the subject application or its progeny are filed. However, it should be understood that the availability of a deposit does not constitute a license to practice the subject invention in derogation of patent rights granted by governmental action.

### Definitions:

In general, the terms and phrases used herein have their art-recognized meaning, which can be found by reference to standard texts, journal references, and context known to those skilled in the art. The following definitions are provided to clarify their specific use in context of the disclosure.

It is to be understood that the fungal strain used in accordance with the methods of the invention may be Metarhizium anisopliae DSM 3884 or Metarhizium anisopliae DSM 3885; however, the fungal strain may also be a culture of strain having properties substantially similar to the above mentioned isolated and deposited strains. Preferred properties include those properties of an entomopathogenic fungus capable of infecting and consuming arthropod cadavers as an arthropod pathogen.

As used herein, “pests” can mean any arthropod whose existence it can be desirable to control.

For purposes of simplicity, the term “insect” shall be used in this application; however, it should be understood that the term “insect” refers, not only to insects of the scientific classification (class) insecta such as cockroaches and ants, but also to mites, spiders, and other arachnids, and like invertebrates.

As used herein, the terms “effective amount”, “effective concentration”, or “effective dosage” are defined as the amount, concentration, or dosage of entomopathogenic fungi sufficient to cause infection in the insect which will then lead to the reduction or elimination of odors emitted by dead insects or horizontal transmission in the insect colony. The actual effective dosage in absolute value depends on factors including, but not limited to, the mortality rate of the target insects relative to the rate at which Metarhizium anisopliae is able to infect the insect and propagate within the cadaver while excluding other microorganisms, synergistic or antagonistic interactions between the other active or inert ingredients which may increase or reduce the activity of Metarhizium anisopliae, the inherent susceptibility of the lifestage and species of insect, and the stability of the Metarhizium anisopliae in formulations.

The compositions of the present invention may further comprise one or more agents capable of killing insects. Such agents include, but are not limited to, baits, sprayable and dustable formulations containing the active ingredients: boric acid, abamectin, fipronil, hydramethylnon, indoxacarb, and imidacloprid.

Although the compositions can be used as raw materials, in one embodiment the composition is placed in a container, or trap, suitable for household and/or industrial use. Such containers include, but are not intended to be limited to, metal and plastic cans, boxes, traps, plastic containers, vats, and other enclosed containers containing one or more orifices for the entry and possible exit of the pests but otherwise generally contain the composition for protection against human or other animal exposure.

In another embodiment, the composition further includes a pest attractant. Such attractants may include, but are not limited to, food, food aromas, and pheromones

In another embodiment, the composition may be added to one or more commercial products capable of killing pests. Examples of such commercial products may include, but are not limited to:

Roach Prufe®; Hot Shot Max Attrax®; Roach Powder Avert®; Niban®; Stapleton's Magnetic Roach Food®; Maxforce®; Combat®; Maxforce Siege®; Hot Shot Maxattrax®; Ultra Brand Nest Destroyer Roach Bait®; and Pre-Empt Professional Cockroach Gel Bait®.

### EXAMPLES

The following examples are provided for illustrative purposes and are not intended to limit the scope of the invention as claimed herein. Any variations in the exemplified examples which occur to the skilled artisan are intended to fall within the scope of the present invention.

### Example 1

### Experimental Materials and Methods

German cockroach (Blatella germanica) nymphs were obtained from Benzon Research and kept in a cold room until time of delivery to the experimental arenas. Upon delivery, the cockroach nymphs remained in a cold room until the bioassay was performed. Once the bioassay was initiated, a single German cockroach nymph was added to a predetermined diet cup and all cockroaches were provided with moisture and crushed dog food as a food source. The cockroaches were exposed to moisture by placing a florists' foam circle at the bottom of a diet cup and saturating it with deionized water (diH₂O). The food source was placed in a 2.0 mL microcentrifuge cap to prevent the food from becoming saturated by the diH₂O in the florists' foam circle.

### Procedures

Specifically, the bioassay included subjecting the cockroach nymphs to an array of treatments. The treatments included a control, a treatment with 1×10⁵ conidia/mL Met52, a treatment with 1×10⁶ conidia/mL Met52, a treatment with 1×10⁷ conidia/mL Met52, a treatment with 1×10⁸ conidia/mL Met52, a treatment with cockroach bait only, a treatment with cockroach bait in combination with 1×10⁷ conidia/mL Met52, a treatment with cockroach bait in combination with 10% (w/w) Met52 spore powder (USEPA Registration No.: 70127-7), a treatment with cockroach bait in combination with 1% (w/w) Met52 spore powder, a treatment with dog food treated with 1% (w/w) Met52 spore powder, a treatment with dog food treated with 3% (w/w) Met52 spore powder, and a treatment with dog food treated with 10% (w/w) Met52 spore powder. There were three (3) replicates of ten (10) individually caged cockroach nymphs used for each treatment.

In treatments wherein the fungal spore was used exclusively, a conidial solution was added directly to the foam circle until saturation was reached. This procedure was also used when cockroach bait was used in combination with 1×10⁷ conidia/mL Met52. For the remaining treatments, the florists' wet foam disk was saturated with diH₂O.

For treatments wherein the Met52 spore powder was used in combination with a commercially available bait station, the bait station was cut open and the bait was removed, crushed, and placed in a 2.0 mL microcentrifuge cap. The spore powder was then combined with the crushed bait. Specifically, 0.6 g of spore powder was added into 6.17 g of crushed bait to produce the 10% spore powder bait. To produce the 1% spore powder, 0.48 g of the 10% spore powder bait was added to 4.86 g of bait.

For treatments wherein dog food was combined with spore powder, the food source, was treated directly with the spore powder. Three (3) treatments were prepared. Specifically, approximately 0.1 g of spore powder was combined with approximately 9.9 g of crushed dog food, approximately 0.3 g of spore powder was combined with approximately 9.7 g of crushed dog food, and approximately 1.0 g of spore powder was combined with approximately 9.0 g of crushed dog food approximating 1%, 3% and 10% of spore powder to total weight respectively.

Each of the diet cups were capped with a paperboard lid to allow for sufficient ventilation while preventing excessive moisture loss. The cockroach nymphs were incubated in the diet cups at room temperature over a period of fourteen (14) days and mortality was evaluated daily. All nymphs were provided with sufficient moisture and dog food diet over the course of the bioassay. Insects that died during the fourteen (14) day bioassay were surface sterilized and transferred to a ninety-six (96) well plate containing 1.5% water agar. The well plate was covered with parafilm to maintain high humidity to monitor for sporulation of the fungal strains.

### Results

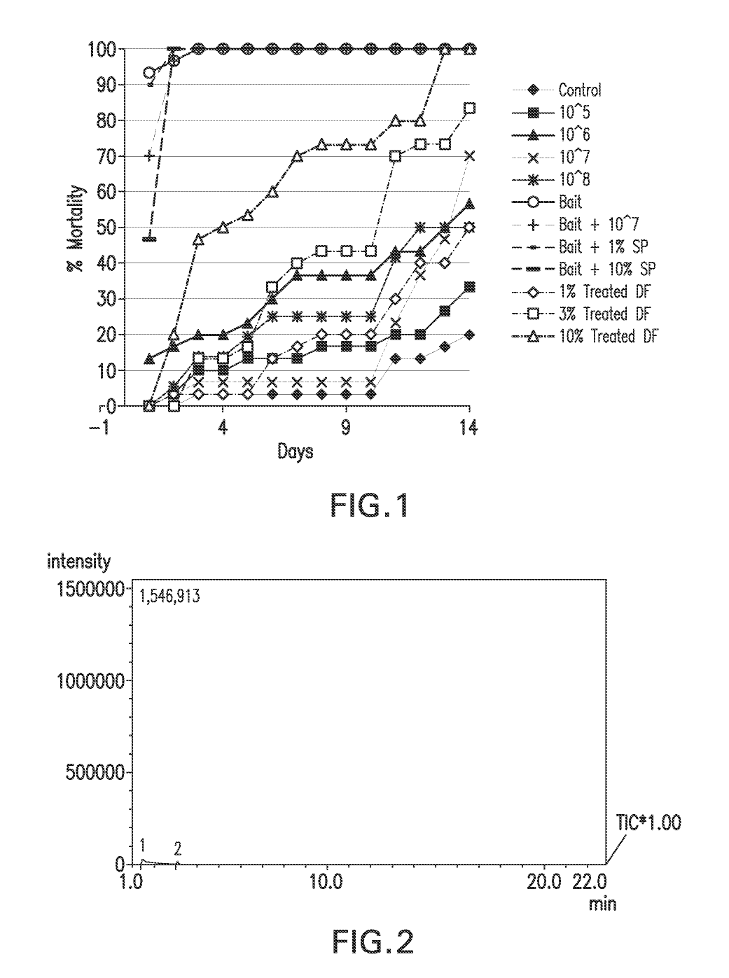

Referring to FIG. 1, after fourteen (14) days, only approximately 20% of nymphs designated as controls were dead. In contrast, after approximately three (3) days, 100% of nymphs subjected to treatments of bait only, bait in combination with 10% (w/w) Met52 spore powder, bait in combination with 1% (w/w) Met52 spore powder, and bait in combination with 1×10⁷ conidia/mL Met52 were dead.

Nymphs subjected to dog food treated with 10% spore powder expressed a percent mortality ranging from 0% mortality to 100% mortality after approximately twelve (12) days. Nymphs subjected to dog food treated with 3% spore powder exhibited a percent mortality ranging from 0% mortality to approximately 82% mortality after approximately fourteen (14) days. Nymphs subjected to treatments of 1×10⁷ conidia/mL Met52 exhibited a percent mortality ranging from 0% mortality to approximately 70% mortality after approximately fourteen (14) days. Nymphs subjected to treatments of 1×10⁶ conidia/mL Met52 exhibited a percent mortality ranging from 0% mortality to approximately 56% mortality after approximately fourteen (14) days. Nymphs subjected to treatments of 1×10⁸ conidia/mL Met52 exhibited a percent mortality ranging from 0% mortality to approximately 50% mortality after approximately fourteen (14) days. Nymphs subjected to dog food treated with 1% spore powder exhibited a percent mortality ranging from 0% mortality to approximately 50% mortality after approximately fourteen (14) days. Finally, nymphs subjected to treatments of 1×10⁵ conidia/mL Met52 exhibited a percent mortality ranging from 0% mortality to approximately 33% mortality after approximately fourteen (14) days.

A blind two choice smell test was conducted between sporulating nymph cadavers exhibiting frank mycosis from M. anisopliae treatments and cadavers that were treated using only cockroach bait. The headspace above the aforementioned cadaver groups was sampled and evaluated using gas chromatography-mass spectrometry (GC-MS). Specifically, a 50/30 μm divinylbenzene/Carboxen/polydimethylsiloxane (DVB/CAR/PDMS) solid phase micro-extraction (SPME) fiber (Supelco) was introduced into the headspace of vials (pre-equilibrated for 5 minutes at 50° C.) using a Combi Pal AOC 5000 autosampler (CTC Analytics). Extraction was carried out for 30 minutes at 50° C. Following extraction, the fiber was immediately introduced into a Shimadzu 2010-S gas chromatograph (GC) equipped with Siltek split/splitless inlet liner (Restek) and an Equity-5 fused silica column (30 m×0.25 mm×0.25 μm film thickness; Sigma-Aldrich) connected to an electron impact quadropole mass spectrometer (MS) system. The injection port temperature was set to 250° C. The column was run at 50° C. for 1 minute and then 10° C./min to 270° C. for a total run time of 23 minutes. Two blank desorptions were performed prior to the first sample to free the fiber of analyte. The GC was operated with a split of 5 mL/min and purge of 0.5 mL/min. Grade 5 helium was used as the carrier gas (1 mL/min column flow) and the MS ion source temperature was set to 180° C. The interface was set to 200° C. and scan mode was used (m/z 40-400). Peak areas were calculated with GC/MS solution software (Shimadzu) and the compounds were identified by comparing obtained spectra to a standard library (NIST Mass Spectral Search Program).

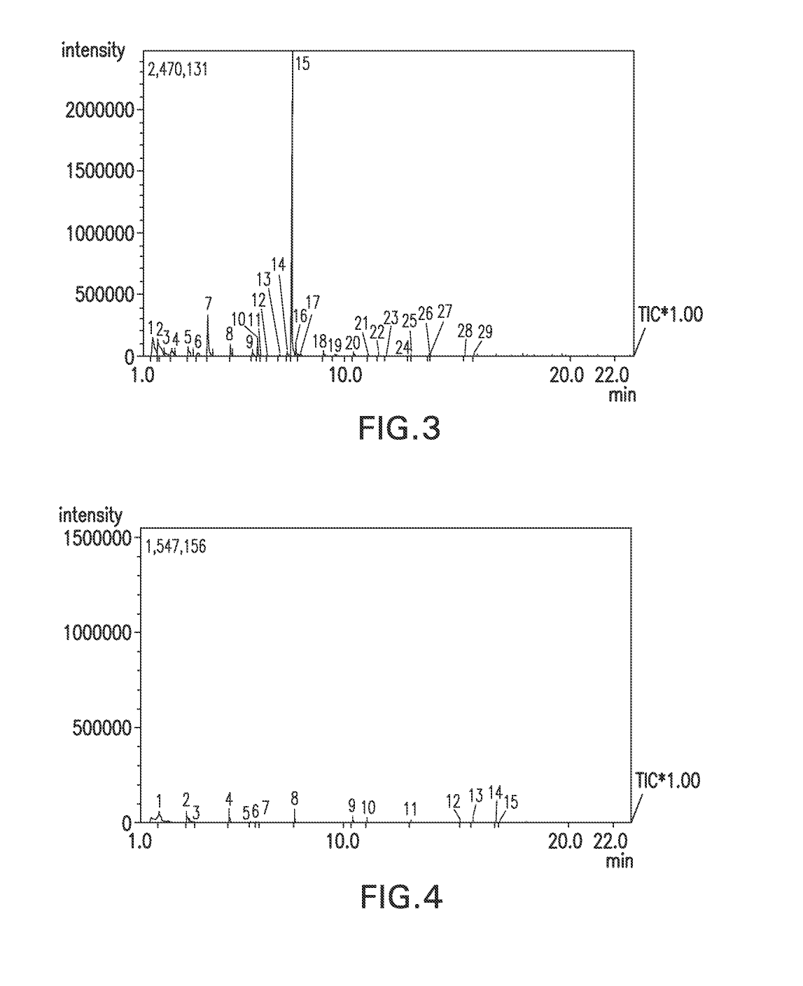

Referring to FIGS. 2-4, the GC-MS results indicate that odors associated with decaying cockroach cadavers are quantitatively reduced when cockroach cadavers exhibited mycosis resulting from bait placed in combination with or treated with M. anisopliae (Met52). Specifically, FIG. 2 is a GC-MS test illustrating volatiles produced from a blank sample. As expected, there are no peaks of interest. FIG. 3 is a GC-MS illustrating the volatiles produced from a sample including cockroach cadavers. FIG. 3 includes twenty-nine (29) total peaks with the most intense peaks being peaks 7 and 15. Peaks 7 and 15 are identified as compounds dimethyl sulfide and phenol respectively. The compounds associated with peaks 7 and 15 generally have odors that are considered to be objectionable and unpleasant smelling. FIG. 4 is a GC-MS illustrating the volatiles produced from a sample including cockroach cadavers sporulating with Met52. In stark contrast to the results shown in FIG. 3, the results shown in FIG. 4 demonstrate that treatment with Met52 drastically reduced not only the total number of volatiles emitted by the cockroach cadavers but the overall intensity of the volatiles as well. Specifically, when Met52 was placed in combination with the cockroach bait, only fifteen (15) total peaks were observed with the most intense peaks being peaks 4 and 8 respectively. In short, there was a clear separation of these treatments with the insects sporulating with Met52 causing significantly less odor, a very different spectrum of volatile compounds, and total peak area of 10× lower than when cockroach bait was used exclusively. Only nine (9) of the twenty-nine (29) peaks identified from volatiles of the nonsporulating cadavers were in common with volatiles from sporulating cadavers.

In addition to the results obtained in FIGS. 2-4 by GC-MS, further tests were conducted wherein thirteen (13) individual participants were asked to decide whether cockroach cadavers treated with Met52 emitted fewer odors than cockroach cadavers treated with bait only. Twelve (12) out of thirteen (13) individuals identified the sporulating cadavers to have fewer odors than the nonsporulating caver confirming the results obtained in the GC-MS experiments.

### Example 2

### Experimental Materials and Methods

Two species of cockroach, B. germanica and B. orientalis, were used to perform a horizontal transmission assay. Sporulated cockroach cadavers from each of the aforementioned species were treated with seven different treatments to investigate the compatibility of an emulsifiable concentrate formulation (EC) (USEPA Registration No.: 70127-10) and technical grade powder (TGP) (USEPA Registration No.: 70127-7) with the active ingredient in Combat Roach Traps®, 0.03% Fipronil. The EC is a combination of the Metarhizium anisopliae spores suspended in oil containing emulsifiers so that the EC can be dispersible in water. The TGP is Metarhizium anisopliae spores exclusively.

Large and small roach traps from Combat Fast Acting Roach Formula® (0.03% Fipronil) were obtained for combination and testing with EC (approximately 4×10⁷ viable conidia/mL suspension) and TGP. The EC was diluted from an approximately 4×10⁹ viable conidia/mL suspension to an approximate 4×10⁹ viable conidia/mL suspension by adding 2 mL of 4×10⁹ viable conidia/mL suspension into 198 mL of sterile ddH₂0 for an approximate 1:100 dilution. Additional materials included, Crisco®, Vaseline®, deionized water (diH₂O), petri dishes, and florists' wet foam. Tested species of cockroach were received from Benzon Research.

### Procedures

Each of the aforementioned cockroach species were exposed to one of seven possible treatments. Specifically, the treatments included exposing the cockroach species to a control of diH₂O, 2 mL/L (approximately 10⁷ conidia/mL) Saturated Wet Foam (SWF), a bait trap exclusively, bait in combination with SWF (2 mL of EC/L of diH₂O), an EC swab placed directly into the entrance of the Bait Trap (2 mL of EC/L of diH₂O), 11% (by weight) of spore powder in combination with Crisco® (approximately 2.75 g of spore powder into approximately 22.40 g of Crisco®), and 11% (by weight) of spore powder in combination with Vaseline® (approximately 2.75 g of spore powder into approximately 22.24 g Vaseline).

For B. germanica, petri dishes were lined with florists' wet foam. The florists' wet foam was treated according to the aforementioned experimental materials and methods. Specifically, the florists' wet foam in each petri dish was saturated with approximately 8.5 mL to 9 mL of diH₂O for the controls and approximately 8.5 mL to 9.0 mL of 4×10⁷ conidia/mL for the treated dishes. There was no standing water or solution in the dishes following treatment. Small roach traps were added to petri dishes treatments which required a trap. Three (3) small holes were perforated into the lid of the petri dish to provide adequate ventilation and gas exchange for the insects. A food source was not added to any of the petri dishes. Following appropriate preparation of the petri dishes, B. germanica cockroaches were asphyxiated with CO₂ and then a mix of approximately ten (10) adult and nymph cockroaches were added to each petri dish in triplicate. Morality and sporulation of the B. germanica cockroaches were monitored over a sixteen (16) day period.

For B. orientalis, sterlite plastic Tupperware® containers were lined with florists' wet foam. The florists' wet foam was treated according to the aforementioned experimental materials and methods. Specifically florists' wet foam was saturated with either diH₂O or 4×10⁷ conidia/mL (2 mL of EC/L of diH₂O). For treatments without cockroach traps, 50 to 52 mL was required to saturate the wet foam, whereas for containers having traps only required approximately 20 mL to saturate the wet foam as the traps covered a portion of the container floor. There was no standing water or solution in the dishes following treatment. Holes were not perforated into the lids of each container and a food source was not added to any of the containers. Following appropriate preparation of the containers, B. orientalis cockroaches were asphyxiated with CO₂ and a mix of approximately seven (7) adult and nymph cockroaches were added to each container. Tests were conducted as a single replicate. Mortality and sporulation of the B. orientalis cockroaches were monitored over a sixteen (16) day period.

### Results

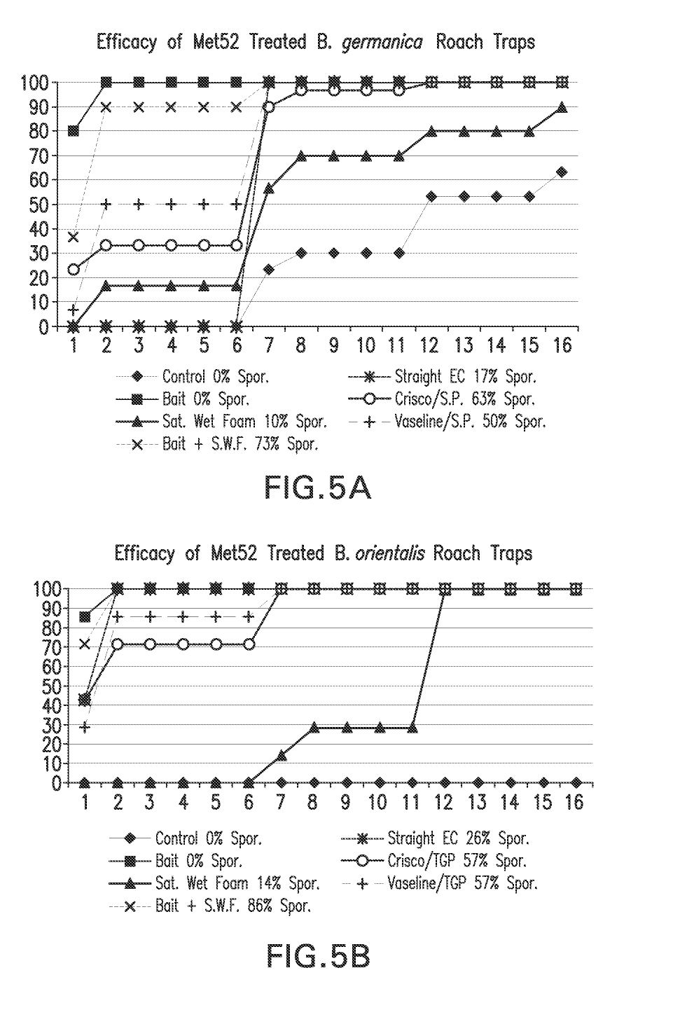

Referring to FIGS. 5A-5B, the efficacy of Met52 when placed in combination with roach traps was observed as a function of percent mortality over time for each of B. germanica and B. orientalis. FIGS. 5A-5B also illustrate the percent sporulation.

FIG. 5A illustrates that B. germanica cockroaches designated as controls exhibited a percent mortality ranging from 0% mortality to approximately 62% mortality after approximately sixteen (16) days. In contrast, B. germanica cockroaches subjected to a bait trap exclusively exhibited a percent mortality ranging from approximately 80% mortality to 100% mortality after approximately a day and a half. B. germanica cockroaches subjected to bait in combination with SWF exhibited d a percent mortality ranging from approximately 38% mortality to 100% mortality after approximately six (6) days. B. germanica cockroaches subjected to an EC swab placed directly into the entrance of a bait trap exhibited a percent mortality ranging from 0% mortality to 100% mortality after approximately six (6) days. B. germanica cockroaches subjected to spore powder in combination with Vaseline° exhibited a percent mortality ranging from approximately 8% mortality to 100% mortality after approximately six (6) days. B. germanica cockroaches subjected to spore powder in combination with Crisco® exhibited a percent mortality ranging from approximately 21% mortality to 100% mortality after approximately twelve (12) days. Finally, B. germanica cockroaches subjected to SWF with 2 mL of EC/L of diH₂O exhibited a percent mortality ranging from 0% mortality to approximately 88% mortality after sixteen (16) days.

For B. germanica cockroaches, neither the control nor exposure to the bait exclusively caused sporulation. In contrast, approximately 73% of B. germanica cockroaches exposed to bait in combination with SWF sporulated, approximately 63% of B. germanica cockroaches exposed to spore powder in combination with Crisco® sporulated, approximately 50% of B. germanica cockroaches exposed to spore powder in combination with Vaseline® sporulated, approximately 17% of B. germanica cockroaches exposed to an EC swab placed directly into the entrance of a bait trap sporulated, and approximately 10% of B. germanica cockroaches exposed to SWF with 2 mL of EC/L of diH₂O sporulated.

FIG. 5B illustrates that B. orientalis cockroaches designated as controls exhibited a 0% mortality after approximately sixteen (16) days. In contrast, B. orientalis cockroaches subjected to a bait trap exclusively exhibited a percent mortality ranging from approximately 85% mortality to 100% mortality after approximately one and a half (1.5) days. B. orientalis cockroaches subjected to bait in combination with SWF with 2 mL of EC/L of diH₂O exhibited a percent mortality ranging from approximately 71% mortality to 100% mortality after approximately one and a half (1.5) days. B. orientalis cockroaches subjected to an EC swab placed directly into the entrance of a bait trap exhibited a percent mortality ranging from approximately 42% mortality to 100% mortality after approximately one and a half (1.5) days. B. orientalis cockroaches subjected to spore powder in combination with Crisco® exhibited a percent mortality ranging from approximately 42% mortality to 100% mortality after approximately six and a half (6.5) days. B. orientalis cockroaches subjected to spore powder in combination with Vaseline® exhibited a percent mortality ranging from approximately 28% mortality to 100% mortality after approximately six and a half (6.5) days. Finally, B. orientalis cockroaches subjected to SWF with 2 mL of EC/L of diH₂O exhibited a percent mortality ranging from 0% mortality to approximately 100% mortality after approximately eleven and a half (11.5) days.

For B. orientalis cockroaches, neither the control nor exposure to the bait exclusively caused sporulation. In contrast, approximately 86% of B. orientalis cockroaches exposed to bait in combination with SWF sporulated, approximately 57% of B. orientalis cockroaches exposed to spore powder in combination with Crisco® sporulated, approximately 57% of B. orientalis cockroaches exposed to spore powder in combination with Vaseline® sporulated, approximately 26% of B. orientalis cockroaches exposed to an EC swab placed directly into the entrance of a bait trap sporulated, and approximately 14% of B. orientalis cockroaches exposed to SWF sporulated.

### Example 3

### Experimental Materials and Methods

A horizontal transmission assay was performed to confirm that the semi-social behavior of cockroaches can be exploited such that a sporulated cadaver can effectively transmit Met52 fungal spores to a representative population of cockroaches. In the assay, approximately ten (10) B. germanica cockroaches and approximately ten (10) B. orientalis cockroaches were made to cohabitate with one another to simulate a small scale colony.

### Procedures

For B. germanica cockroaches, the bottoms of petri dishes were lined with florists' wet foam and saturated with deionized water (diH₂O). Ten (10) to twelve (12) B. germanica cockroaches were asphyxiated via CO₂ and placed in petri dishes with a single cockraoch cadaver set aside from the Odor Control Assay/GC-MS of Example 1. This procedure was replicated six (6) times. None of the cadavers used were used in GC/MS experiment. Each of the petri dishes was parafilmed and the assay was performed three (3) times with crushed dog food and three (3) times without crushed dog food. No controls were used for this assay.

For B. orientalis cockroaches, the bottoms of sterlite Tupperware® containers were lined with florists' wet foam and saturated with diH₂O. Ten (10) B. orientalis cockroaches were asphyxiated via CO₂ and placed in containers with a sporulated cockroach cadaver. Each of the containers was closed with a lid and the assay was performed three (3) times with crushed dog food and three (3) times without crushed dog food. No controls were used for this assay.

### Results

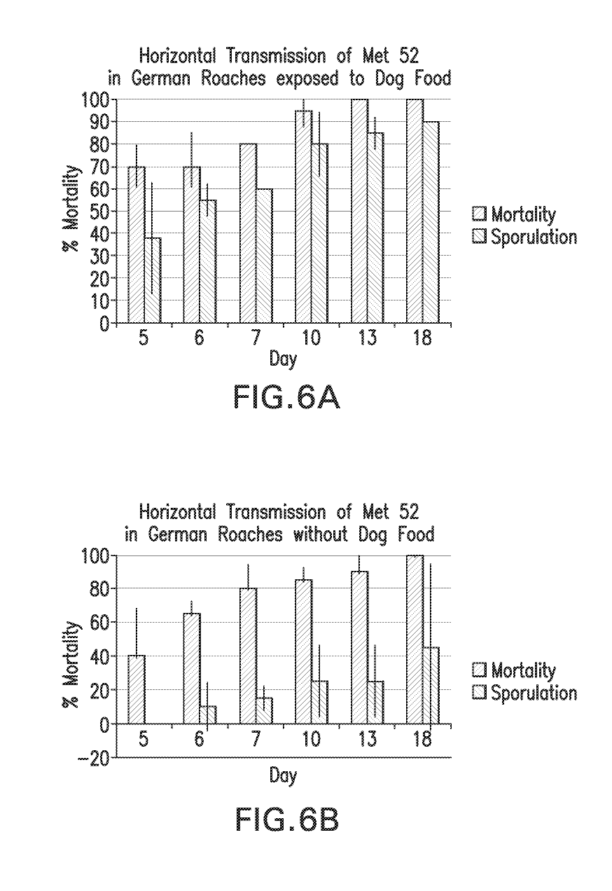

FIGS. 6A-6B are bar graph representations of the effectiveness of Met52 to be horizontally transmitted among B. germanica cockroaches when in the presence and absence of dog food as a food source. More specifically, FIGS. 6A-6B demonstrate the efficacy of Met52 to be horizontally transmitted among a population of B. germanica cockroaches as a function of percent mortality over time.

Referring to FIGS. 6A-6B, a positive correlation exists between percent mortality and percent sporulation. In particular, as the percentage of Met52 sporualting cadavers increases in a population of B. germanica cockroaches, the overall percent mortality increases among that population of cockroaches as well. Specifically, as represented in FIG. 6A, when dog food was present, the percent mortality was approximately 70% after approximately five (5) days and reaching 100% mortality on or about day seventeen (17). As illustrated in FIG. 6B, when dog food was absent, the percent mortality was approximately 40% after approximately five (5) days and reaching 100% mortality on or about day seventeen (17).

Comparing the results of FIG. 6A to the results of FIG. 6B, it becomes apparent that this statistical correlation is stronger when the cockroaches are exposed to dog food in addition to Met52 sporulated cadavers. Notwithstanding the strength of the correlation, FIGS. 6A-6B clearly demonstrate that as the percentage of cadavers sporulating with Met52 increases in a population of B. germanica cockroaches, the percent mortality increases among that population of cockroaches as well. FIGS. 6A-6B further demonstrate that the semi-social behavior of cockroaches can be exploited to horizontally transmit Met52 spores among a population of B. germanica cockroaches. The data presented in FIGS. 6A-6B will support the use of Met52 in cockroach trap applications to reduce odors associated with decaying cockroach cadavers. There was no apparent avoidance behavior by the cockroaches to the sporulating cadaver and live cockroaches could often be seen in close association with the sporulating cadavers. It is worth noting that the pathogenicity of conidia from these cadavers is apparently high given the fast mortality rate and high sporulation rate of the cockroaches relative to previous experiments where the source of conidia was either spore powder or EC. It is well documented in the literature that conidia produced in vivo are often more pathogenic than those produced in vitro, which would tend to favor horizontal transmission in the colony once the cockroaches are initially killed by the chemical pesticide.

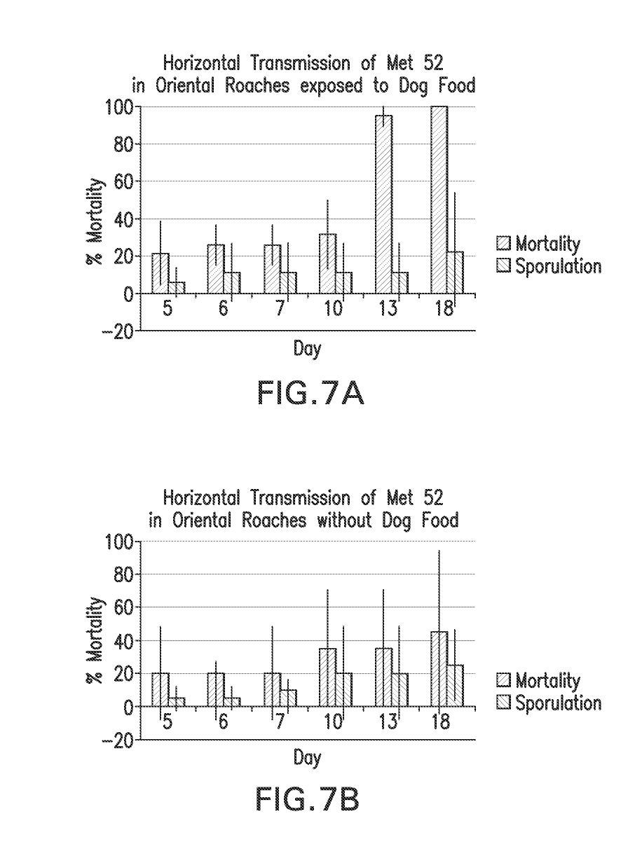

FIGS. 7A-7B are bar graph representations of the effectiveness of Met52 to be horizontally transmitted among B. orientalis cockroaches when in the presence and absence of dog food as a food source. More specifically, FIGS. 7A-7B demonstrate the efficacy of Met52 to be horizontally transmitted among a population of B. orientalis cockroaches as a function of percent mortality over time.

Referring to FIGS. 7A-7B, a positive correlation exists between percent mortality and percent sporulation. In particular, as the percentage of Met52 sporualting cadavers increases in a population of B. orientalis cockroaches, the overall percent mortality increases among that population of cockroaches as well. Specifically, as represented in FIG. 7A, when dog food was present, the percent mortality was approximately 21% after approximately five (5) days and reaching 100% mortality on or about day seventeen (17). As illustrated in FIG. 7B, when dog food was absent, the percent mortality was approximately 40% after approximately five (5) days and reaching 100% mortality on or about day seventeen (17).

Comparing the results of FIG. 7A to the results of FIG. 7B, the correlation is stronger when the cockroaches are exposed to dog food in addition to Met52 sporulated cadavers. Notwithstanding the strength of the correlation, FIGS. 7A-7B also demonstrate that as the percentage of cadavers sporulating with Met52 increases in a population of B. orientalis cockroaches, the percent mortality increases among that population of cockroaches as well. FIGS. 7A-7B further demonstrate that the semi-social behavior of cockroaches can be exploited to horizontally transmit Met52 spores among a population of B. orientalis cockroaches. The data presented in FIGS. 7A-7B will support the use of Met52 in cockroach trap applications to reduce odors associated with decaying cockroach cadavers.

### Example 4

### Experimental Materials and Methods

A second horizontal transmission assay was performed to confirm that the semi-social behavior of cockroaches can be exploited such that a sporulated cadaver can effectively transmit Met52 fungal spores to a representative population of cockroaches under optimal growth conditions for the Met52 fungus; namely, under conditions of high humidity. In the assay, approximately ten (10) to fifteen (15) B. germanica cockroach nymphs were made to cohabitate with one another to simulate a small scale colony.

### Procedures

Two-hundred fifty (250) B. germanica cockroach nymphs were acquired from Benzon Research. The bottoms of petri dishes were lined with florists' wet foam and saturated with deionized water (diH₂O). Ten (10) to fifteen (15) B. germanica cockroach nymphs were asphyxiated via CO₂ and placed in petri dishes with a single sporulated cockroach cadaver from the horizontal transmission study disclosed and described in Example 3. In one petri dish, forty (40) roaches were placed together with a single sporulated cockroach cadaver. Each of the petri dishes was parafilmed and the assay was performed twenty (20) times with crushed dog food placed in small vial lids as a food source. No controls were used for this assay.

### Results

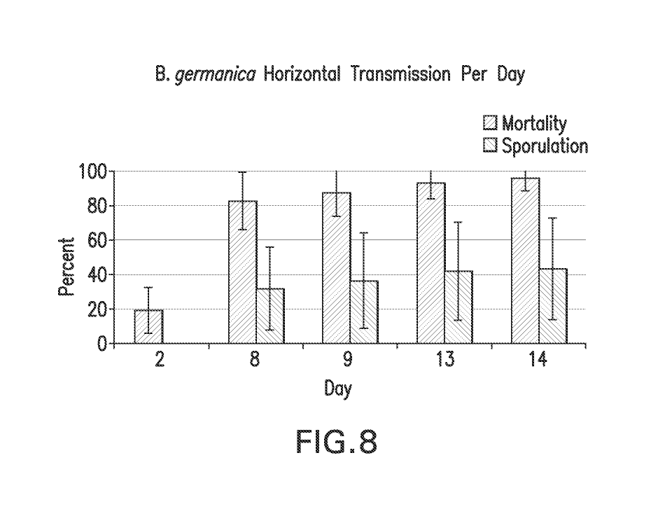

FIG. 8 is a bar graph representation of the effectiveness of Met52, under optimal growth conditions, to be horizontally transmitted among B. germanica cockroaches in the presence of dog food as a food source. More specifically, FIG. 8 demonstrates the efficacy of Met52 to be horizontally transmitted among a population of B. germanica cockroaches as a function of percent mortality over time.

Referring to FIG. 8, a positive correlation exists between percent mortality and percent sporulation. In particular, as the percentage of Met52 sporualting cadavers increases in a population of B. germanica cockroaches, the overall percent mortality increases among that population of cockroaches as well. Specifically, as represented in FIG. 8, the percent mortality was approximately 20% after approximately five (5) days and approximately 95% mortality on or about day fourteen (14).

Under optimal conditions, the larger study of B. germanica cockroaches and their ability to transmit Met52 spores from a sporulating cadaver to other members of the population proved effective. The larger number of repetitions performed in this assay caused a higher standard deviation with respect to the percent of the population that sporulated. The data can be extrapolated over the course of two weeks to show that, on average, 40% of the cockroaches introduced to a single sporulated cadaver were infected, consumed by Met52 hyphal growth, and then positively identified as being sporulated by Met52.

The present invention is described by the following numbered paragraphs:
1. A composition for reducing or eliminating malodors produced by a decaying arthropod cadaver comprising an effective amount of one or more entomopathogenic fungi.
2. The composition of paragraph 1 wherein said entomopathogenic fungi is selected from the group consisting of Metarhizium spp., Beauveria spp., Paecilomyces spp, Lecanicillium spp., and Hirsutella spp.
3. The composition of paragraph 1 wherein said entomopathogenic fungus is Metarhizium anisopliae.
4. The composition of paragraph 3 wherein said entomopathogenic fungus is DSM 3884, DSM 3885, or a mixture thereof.
5. The composition of paragraph 1 wherein said composition further comprises a chemical pesticide.
6. The composition of paragraph 5 wherein said chemical pesticide is a bait formulation, a sprayable formulation, and a dustable formulation.
7. The composition of paragraph 6 wherein said chemical pesticide includes an active ingredient, said active ingredient being selected from the group consisting of boric acid, abamectin, fipronil, hydramethylnon, indoxacarb, and imidacloprid.
8. The composition of paragraph 1 wherein said arthropod is a cockroach.
9. The composition of paragraph 8 wherein said cockroach is a German cockroach Blatella germanica, a brown banded cockroach Supella longipaloa, an Oriental cockroach Blatta orientalis, a smoky brown cockroach Periplaneta fuliginosa, an American cockroach Periplaneta Americana, a Turkenstan cockroach Blatta lateralis, and a field cockroach Blatta vaga.
10. A method for reducing or eliminating malodors produced by decaying arthropod cadavers comprising:
   (a) preparing a composition having an effective amount of one or more entomopathogenic fungi; and
   (b) exposing said composition to a target arthropod pest.
11. The method of paragraph 10, wherein the entomopathogenic fungi is selected from the group consisting of Metarhizium spp., Beauveria spp., Paecilomyces spp, Lecanicillium spp., and Hirsutella spp.
12. The method of paragraph 11 wherein said entomopathogenic fungus is Metarhizium anisopliae.
13. The method of paragraph 12 wherein said entomopathogenic fungus is DSM 3884, DSM 3885, or a mixture thereof.
14. The method of paragraph 10 wherein said target arthropod pest is exposed to said composition by placing said composition in a trap.
15. The method of paragraph 10 wherein said target arthropod pest is exposed to said composition by combining said composition with a food source.
16. The method of paragraph 10 wherein said target arthropod pest is exposed to said composition by combining said composition with a chemical pesticide.
17. The method of paragraph 16 wherein said chemical pesticide is a bait formulation, a sprayable formulation, and a dustable formulation.
18. The method of paragraph 17 wherein said chemical pesticide includes an active ingredient, said active ingredient being selected from the group consisting of boric acid, abamectin, fipronil, hydramethylnon, indoxacarb, and imidacloprid.
19. The method of paragraph 10 wherein said target arthropod pest is one or more cockroaches.
20. The method of paragraph 19 wherein said cockroach is a German cockroach Blatella germanica, a brown banded cockroach Supella longipaloa a Oriental cockroach Blatta orientalis, a smoky brown cockroach Periplaneta fuliginosa an American cockroach Periplaneta Americana, a Turkenstan cockroach Blatta lateralis, and a field cockroach Blatta vaga.
21. An insect trap comprising a chamber capable of attracting an insect and one or more compositions for reducing or eliminating malodors produced by a decaying arthropod cadaver comprising an effective amount of one or more entomopathogenic fungi.
22. The insect trap of paragraph 21 wherein said entomopathogenic fungi is selected from the group consisting of Metarhizium spp., Beauveria spp., Paecilomyces spp, Lecanicillium spp., and Hirsutella spp.
23. The composition of paragraph 21 wherein said entomopathogenic fungus is Metarhizium anisopliae.
24. The composition of paragraph 23 wherein said entomopathogenic fungus is DSM 3884, DSM 3885, or a mixture thereof.
25. The composition of paragraph 21 wherein said composition further comprises a chemical pesticide.
26. The composition of paragraph 25 wherein said chemical pesticide is a bait formulation, a sprayable formulation, and a dustable formulation.
27. The composition of paragraph 26 wherein said chemical pesticide includes an active ingredient, said active ingredient being selected from the group consisting of boric acid, abamectin, fipronil, hydramethylnon, indoxacarb, and imidacloprid.

It will be understood that the Specification and Examples are illustrative of the present invention and that other embodiments within the spirit and scope of the invention will suggest themselves to those skilled in the art. Although this invention has been described in connection with specific forms and embodiments thereof, it would be appreciated that various modifications other than those discussed above may be resorted to without departing from the spirit or scope of the invention as defined in the appended claims. For example, equivalents may be substituted for those specifically described, and in certain cases, particular applications of steps may be reversed or interposed all without departing from the spirit or scope for the invention as described in the appended claims.

1-27. (canceled)

28. A composition for reducing or eliminating malodors produced by a decaying arthropod cadaver comprising an effective amount of one or more entomopathogenic fungi.

29. The composition of claim 28 wherein said entomopathogenic fungi is selected from the group consisting of Metarhizium spp., Beauveria spp., Paecilomyces spp, Lecanicillium spp., and Hirsutella spp.

30. The composition of claim 28 wherein said entomopathogenic fungus is Metarhizium anisopliae.

31. The composition of claim 30 wherein said entomopathogenic fungus is DSM 3884, DSM 3885, or a mixture thereof.

32. The composition of claim 28 wherein said composition further comprises a chemical pesticide.

33. The composition of claim 32 wherein said chemical pesticide is a bait formulation, a sprayable formulation, and a dustable formulation.

34. The composition of claim 33 wherein said chemical pesticide includes an active ingredient, said active ingredient being selected from the group consisting of boric acid, abamectin, fipronil, hydramethylnon, indoxacarb, and imidacloprid.

35. The composition of claim 28 wherein said arthropod is a cockroach.

36. The composition of claim 35 wherein said cockroach is a German cockroach Blatella germanica, a brown banded cockroach Supella longipaloa, an Oriental cockroach Blatta orientalis, a smoky brown cockroach Periplaneta fuliginosa, an American cockroach Periplaneta Americana, a Turkenstan cockroach Blatta lateralis, and a field cockroach Blatta vaga.

37. A method for reducing or eliminating malodors produced by decaying arthropod cadavers comprising:
(a) preparing a composition having an effective amount of one or more entomopathogenic fungi; and
(b) exposing said composition to a target arthropod pest.

38. The method of claim 37, wherein the entomopathogenic fungi is selected from the group consisting of Metarhizium spp., Beauveria spp., Paecilomyces spp, Lecanicillium spp., and Hirsutella spp.

39. The method of claim 38 wherein said entomopathogenic fungus is Metarhizium anisopliae.

40. The method of claim 39 wherein said entomopathogenic fungus is DSM 3884, DSM 3885, or a mixture thereof.

41. The method of claim 37 wherein said target arthropod pest is exposed to said composition by placing said composition in a trap.

42. The method of claim 37 wherein said target arthropod pest is exposed to said composition by combining said composition with a food source.

43. The method of claim 37 wherein said target arthropod pest is exposed to said composition by combining said composition with a chemical pesticide.

44. The method of claim 43 wherein said chemical pesticide is a bait formulation, a sprayable formulation, and a dustable formulation.

45. An insect trap comprising a chamber capable of attracting an insect and one or more compositions for reducing or eliminating malodors produced by a decaying arthropod cadaver comprising an effective amount of one or more entomopathogenic fungi.

46. The composition of claim 45 wherein said entomopathogenic fungus is DSM 3884, DSM 3885, or a mixture thereof.

47. The composition of claim 45 wherein said composition further comprises a chemical pesticide.
